(19) United States
(12) Patent Application Publication
(10) Pub. No.: US 2024/0287017 A1
(43) Pub. Date: Aug. 29, 2024

(54) A METHOD FOR SYNTHESIZING GLYCOLIDE

(71) Applicant: East China University of Science and Technology, Shanghai (CN)

(72) Inventors: Jinghong ZHOU, Shanghai (CN); Xiaofeng XU, Shanghai (CN); Yueqiang CAO, Shanghai (CN); Wei LI, Shanghai (CN)

(73) Assignee: East China University of Science and Technology, Shanghai (CN)

(21) Appl. No.: 18/569,830

(22) PCT Filed: Jun. 14, 2022
(86) PCT No.: PCT/CN2022/098617

§ 371 Date: Dec. 13, 2023

(30) Foreign Application Priority Data
Jun. 18, 2021 (CN) 202110675326.4

(51) Int. Cl. C07D 319/12; C07C 67/02

(52) U.S. Cl. CPC C07D 319/12; C07C 67/02

(57) ABSTRACT

This invention provides a method for synthesizing glycolide using methyl glycolate as a raw material, comprising two steps as illustrated in the following reaction formulas:

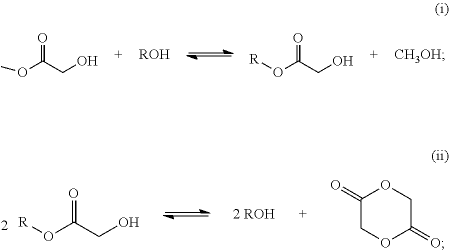

wherein, the ROH represents a straight/branched chain alcohol, acid or ester containing 18-30 carbons and at least one hydroxyl group. The method for glycolide synthesis disclosed in the present invention manages to maintain a low viscosity within the reaction system by introducing a high-boiling alcohol into the reaction system to form a larger alkoxy glycolate exhibiting a significantly lower molecular weight compared to the typical glycolic acid oligomers utilized in the conventional process. Thus the challenges of coking and inefficient heat transfer encountered in traditional glycolide synthesis routes are effectively addressed.

### TECHNICAL FIELD

The present invention relates to new technology for preparing polymer monomers. More specifically, this invention relates to a method for synthesizing glycolide.

### BACKGROUND

The escalating issue of white pollution worldwide has driven a growing need for biodegradable plastics. Consequently, polyglycolic acid (PGA), a noteworthy biodegradable plastic known for its excellent performance in medical and packaging applications, has emerged as a promising solution with significant market potential.

PGA is primarily synthesized through two routes: (1) Direct polycondensation of glycolic acid and (2) Ring-opening polymerization of glycolide. The method of direct polycondensation of glycolic acid, while straightforward, is constrained in its ability to produce high molecular weight PGA, thereby restricting its versatility for various applications. On the other hand, the ring-opening polymerization method, despite its greater complexity, offers precise control over the polymerization degree tailored to meet diverse application needs, enabling it to emerge as the predominant approach for PGA production.

Glycolide, as the monomer for PGA synthesis from ring-opening polymerization, is typically synthesized through two primary routes. One involves depolymerization following the oligomerization of glycolic acid, while the other entails the direct cyclization of methyl glycolate in the presence of catalyst.

The method of two-stage oligomerization-depolymerization for preparing glycolide consists of two steps: (1) Dehydration condensation of glycolic acid to generate glycolic acid oligomers, and (2) Depolymerization of the formed oligomers at high temperatures to produce cyclized glycolide. For example, US Patent U.S. Pat. No. 2,668,162 discloses an oligomerization-depolymerization route, where glycolic acid solution is oligomerized at 175-180° C. under vacuum of 20 kPa, resulting in glycolic acid oligomers. These glycolic acid oligomers are then crushed and depolymerized under conditions of 1.6-2.0 kPa and 270-285° C., at 20 g/h feed rate to yield glycolide.

CN106397389A discloses a process where initially glycolic acid was dehydrated into oligomers with an average molecular weight (Mw) of 5000 g/mol. Subsequently, the glycolic acid oligomers were depolymerized under conditions of 0.4 kPa and 250-280° C., while the glycolide is obtained through distillation.

In CN105218512A, an aqueous solution of glycolic acid is used as the starting material and undergoes oligomerization at 170-190° C. Then the resulting glycolic acid oligomers are introduced into a reactor in batches and undergo depolymerization under conditions of 2.4 kPa and 280-300° C. with resulting glycolide obtained through distillation.

In CN105272958A, crystalline glycolic acid is melted at 90° C. and gradually ramping to 140° C. with tin octoate as a catalyst to undergo oligomerization until no more water evaporates. Then the oligomerization is continued at vacuum of 3 kPa to intensify condensation. The resulting glycolic acid oligomers are further depolymerized at 230-290° ° C. and 0.1-1 kPa to produce glycolide.

In CN112707884A, the introduction of heat-conducting particles made of materials such as mild steel, copper, or alumina serves to enhance heat transfer during the depolymerization of glycolic acid oligomers, effectively mitigating the occurrence of coking. In CN87107549A, glycolic acid is first copolymerized with polyethers of high thermal stability such as polyether glycols with molecular weights ranging from 900 to 3000 at 200° C. and 30 kPa. Subsequently, depolymerization is carried out at 280° C. and 0.9 kPa to yield glycolide.

In recent years, methyl glycolate has emerged as an alternative raw material for producing oligomeric compounds, which are subsequently depolymerized to yield glycolide. For instance, in CN112469759A, a blend of ethylene glycol, dimethyl oxalate, and oxalic acid, not exceeding 1% of the mass of methyl glycolate, is introduced during the methyl glycolate oligomerization process. Furthermore, 5% to 500% of either polyethylene glycol or paraffin, relative to the mass of methyl glycolate, is employed as a viscosity reducer during depolymerization, effectively mitigating issues related to coking. In CN112028868A, glycolic acid oligomers are obtained by gradually raising the temperature from 150 to 210° C. using methyl glycolate as the raw material and stannous octoate as the catalyst. Subsequently, depolymerization occurred at 240-260° ° C. under a pressure of 1.0-1.5 kPa, resulting in the glycolide product.

Nevertheless, in the process of oligomerizing glycolic acid/ester followed by depolymerization to synthesize glycolide, the substantial molecular weight (ranging from 4,000 to 30,000 g/mol) and high viscosity of the intermediate product, glycolic acid oligomers, give rise to issues of inefficient heat transfer and severe coking. This stands as a major technical drawback of the process, prompting researchers to devise various solutions. One such solution is presented in U.S. Pat. No. 5,830,991, which introduced butyl benzyl phthalate as an azeotropic agent to the glycolic acid oligomer and employed polyethylene glycol as a solubilizer. Depolymerization is then carried out at 265˜275° C. and 5 kPa, resulting in an 85% yield of glycolide. Another approach, detailed in CN102712617B, involved the preparation of glycolide by depolymerizing glycolic acid oligomers at 230° ° C. and 2 kPa. This method utilized polyalkylene glycol ether with a boiling point between 280 and 420° C. as an azeotropic agent, polyalkylene glycol, or polyalkylene glycol monoether with a boiling point exceeding 450° C. as co-solvents, and stannous chloride as a catalyst.

In CN1266146C, a glycolic acid aqueous solution is used as raw material and undergoes oligomerization with the introduction of lauryl triglyceride to alleviate the impact of acid impurities. Subsequently, the resulting glycolic acid oligomer is depolymerized using polyethylene glycol diether as an azeotropic agent to yield glycolide which is then co-distilled out of the system with the azeotrope. In CN107868076A, glycolic acid crystalline is used as raw material to synthesize glycolic acid oligomers which are then depolymerized at 270° C. and 1.5 kPa with polyethylene glycol or polyethylene glycol monomethyl ether as solvent. Importantly, the solvent is not co-distilled with the resulting glycolide, thereby producing glycolide with reduced impurity. Although the introduction of a high-boiling-point solvent as azeotropic agent in the preparation of glycolide can enhance its yield, this method does not effectively reduce production cost due to the substantial co-distillation of the azeotropic solvent with the glycolide. This co-distillation consumes huge energy and necessitates additional steps to separate the glycolide from the azeotropic agent.

Patent RU2660652 discloses a method where the copolymerization of glycolic acid with either ethylene glycol or propanetriol at a 17:1 molar ratio with respect to alcohol hydroxyl groups occurs at temperatures between 130-180° C. and yields copolymers with an approximate molecular weight (Mw) of 2000 g/mol. Subsequently, depolymerization takes place at 250-270° C. and under a pressure of 1-2 kPa. The glycolide and polyol are co-distilled out and separated through recrystallization to attain high-purity glycolide. Similarly, a method involving copolymerization with alcohols is employed in the preparation of lactide. Wang Guocai et al. (Guocai Wang, et al., I&ECR, 2018, Vol. 57 (No. 22): 7711-7716) copolymerized lactic acid with pentaerythritol, maintaining a molar ratio of 15:1 with regard to hydroxyl groups. The reaction is carried out at temperatures ranging from 120-140° C. and 3 kPa, yielding copolymers with an approximate Mw of 4000 g/mol. Subsequently, depolymerization was conducted under 210° C. and a pressure of 0.4 kPa, producing lactide with a yield up to 93%. In these approaches, the addition of alcohol serves to limit the growth of the oligomer chain, resulting in lower molecular weight and reduced oligomer viscosity. Consequently, this enhances heat transfer within the system and contributes to an increased yield of the cyclic ester.

Gas-phase direct cyclization to prepare glycolide is a method that allows the direct synthesis of glycolide from glycolic acid (or its esters) in gas phase. For instance, Rik De Clercq et al. (De Clercq et al., ChemCatChem, 2018, Vol. 10 (No. 24): 5649-5655) employed an inert gas, such as nitrogen, as a carrier to entrain vaporized methyl glycolate into the reactor, where glycolide and methanol are produced in the presence of a TiO2/SiO2 catalyst. Methanol vapor is extracted from the upper section of separator while glycolide is obtained from the lower section upon condensation. In CN112010834A, vaporized methyl glycolate at temperatures between 200-400° C. is introduced into a reactor equipped with a tin-containing molecular sieve catalyst. The cyclization reaction occurred at 240-320° C., resulting in the formation of glycolide. However, it's worth noting that the catalysts used in these methods faces challenges related to its lack of industrial viability. This is primarily due to the complexity of catalyst preparation, rapid catalyst deactivation, limited processing capacity, and high energy consumption.

Furthermore, CN1080921A discloses a method for preparing cyclic esters from hydroxy acids and their derivatives. In its Example 53, it describes a procedure for synthesizing lactide from octadecyl lactate. This reaction is conducted at a pressure of 2 Torr and temperatures of approximately 180-190° C. in the presence of catalysts, including stannous octanoate and zinc oxide. Following the reaction, the product is separated by distillation, resulting in a mixture that comprises lactide, 1-octadecanol, octadecyl lactate, and other components, and the lactide content in this mixture reaches up to 48%. It is important to note the presence of an azeotropic phenomenon between lactide and 1-octadecanol within the system.

In the state of the art of glycolide synthesis routes, the two-stage depolymerization-oligomerization method faces a significant challenge due to the high viscosity of the glycolic acid oligomers formed during the oligomerization process. This viscosity issue leads to poor heat transfer, severe degradation, coking, and the accumulation of heavy residues in the reactor, resulting in both low glycolide yield and difficult-to-clean foul. To address this issue, approaches such as introducing co-solvents during oligomerization process have been attempted to mitigate coking, but often necessitates extra expensive solvent separation and purification processes. On the other hand, the gas-phase direct preparation of glycolide is still in the early stages of laboratory research. So far the catalysts developed for this route exhibit limited processing capacity, proneness to deactivate through coking, and other issues such as complexity of catalyst preparation.

From a different perspective, the majority of current research on glycolide synthesis utilizes glycolic acid derived from petroleum as the starting material. Recently, the advancement of coal-to-glycol technology via oxalate hydrogenation has become mature and been commercially applied in China. And the process of synthesizing methyl glycolate through oxalate semi-hydrogenation holds the promise of achieving large-scale production and substantially reduce production costs when compared to methyl glycolate from petroleum. Hence, if a cost-effective and straightforward means of glycolide synthesis using methyl glycolate as the raw material could be developed, it would pave the way for increased production and widespread utilization of PGA materials.

### Invention Description

The object of this invention is to offer a glycolide synthesis method that addresses the shortcomings of current glycolide production techniques, enabling cost-effective large scale production of glycolide.

To attain this objective, the present invention employs the following technical solutions. A glycolide synthesis method is proposed wherein methyl glycolate is utilized as the starting raw material and glycolide is attained through two reaction steps:

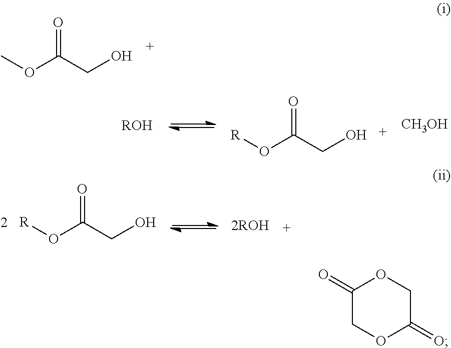

Wherein, ROH represents a straight/branched chain alcohol, acid or ester containing 18-30 carbons and at least one hydroxyl group. The ROH obtained in step (ii) is recycled.

According to the present invention, the said step (i) is conducted with a catalyst at a reaction temperature ranging from 100 to 180ºC.

According to the present invention, the said catalyst is selected from stannous octanoate, stannous chloride, zinc oxide, antimony trioxide, and zinc acetylacetonate.

According to the preferred embodiment of the present invention, the mass ratio of the catalyst to methyl glycolate is in the range of 0.2 to 1.5 wt %.

According to the preferred embodiment of the present invention, the molar ratio of the hydroxyl group present in ROH to methyl glycolate fed in the reaction of step (i) ranges from 1:0.6 to 1:20.

According to the present invention, the said step (ii) is carried out under vacuum distillation, with a pressure ranging from 0.5 to 1.5 kPa and a temperature ranging from 200 to 260° C.

According to the present invention, the boiling point of said ROH is in the range of 330 to 460° C.

The invention also provides a method for synthesizing glycolide wherein methyl glycolate is used as raw material and the glycolide is obtained through the following reaction loop, along with the recyclable ROH:

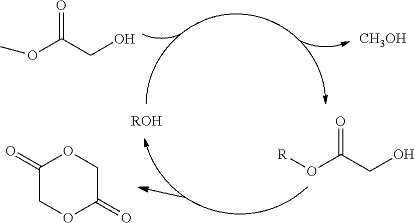

(i) In this step, the raw material methyl glycolate undergoes a transesterification reaction with ROH between 100 to 180 ºC. The esterified product with a larger RO-group is then yielded in high purity as the methanol obtained is distilled out of the system;
(ii) Subsequently, the esterification product of step (i) is heated up to 200 to 260° C. The resulting glycolide is continuously distilled out at a pressure of 0.5 kPa to 1.5 kPa, while ROH is retained in the system for recycling.

Wherein, ROH represents a straight/branched chain alcohol, acid or ester containing 18-30 carbons and at least one hydroxyl group, with a boiling point between 330 and 460° C.

According to the present invention, the said step (i) is carried out with a catalyst selected from stannous octanoate, stannous chloride, zinc oxide, antimony trioxide, and zinc acetylacetonate.

According to the preferred embodiment of the present invention, the mass ratio of the catalyst to methyl glycolate is in the range of 0.2 to 1.5 wt %.

According to the preferred embodiment of the present invention, the molar ratio of the hydroxyl group contained in ROH to methyl glycolate fed in the reaction of step (i) ranges from 1:0.6 to 1:20.

The synthesis method for glycolide in this invention offers numerous advantages over the art:

1. The current invention manages to maintain a low viscosity within the reaction system, effectively addressing the challenges of common coking and inefficient heat transfer encountered in traditional processes. This is achieved by introducing a high-boiling alcohol to transesterified with the starting methyl glycolate, resulting in the formation of a larger alkoxy glycolate exhibiting a significantly lower molecular weight compared to the typical glycolic acid oligomers utilized in conventional procedures.

2. The larger alkoxy glycolate could further convert to glycolide with elevated purity and reduced acid impurity for the formation of linear glycolic acid dimers during this process proves to be challenging. Additionally, the high-boiling alcohols introduced in the process can be efficiently recycled throughout the synthetic pathway.

3. The reaction system employed for glycolide synthesis in this invention is straightforward, readily for industrial scaling, and cost-effective for glycolide production.

### BRIEF DESCRIPTION OF THE DRAWINGS

FIG. 1 is a schematic of the method to synthesize glycolide described in the present invention.

FIG. 2 shows the 1H NMR spectrum of the glycolide prepared in Example 1 of the present invention.

FIG. 3 shows the infrared spectrum of the glycolide prepared in Example 1 of the present invention.

FIG. 4 shows the DSC curves of the glycolide prepared in Example 1 of the present invention.

### DETAILED DESCRIPTION

The method for synthesizing glycolide of the present invention is described in detail exemplified with specific examples. It is essential to note that the following examples are intended only to illustrate the present invention, and are not to be construed as limiting it to any particular embodiment described herein.

All the raw materials and equipment used in the ensuing examples, unless explicitly specified otherwise, are readily obtainable through commercial sources.

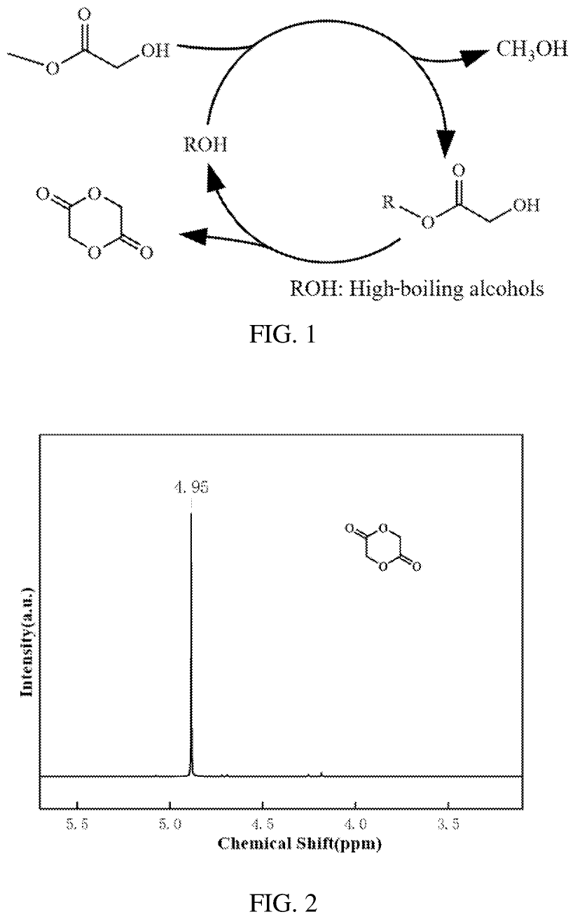

The reaction route of the method in the present invention to synthesize glycolide is shown in FIG. 1. Glycolide is obtained by two reaction steps using methyl glycolate as the raw material as follows:

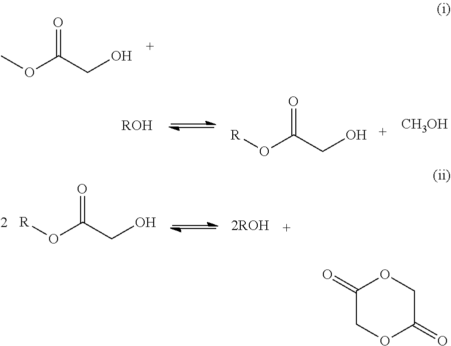

Wherein, ROH represents a straight/branched chain alcohol, acid or ester containing 18-30 carbons and at least one hydroxyl group. The ROH obtained in step (ii) is recycled.

In the current invention the purity of glycolide was determined by internal standard method using a gas chromatography (GC). The GC employed for this analysis is a Linghua GC9890, equipped with a TH-1 column (30 m×0.32 mm×0.5 μm) and a hydrogen flame ionization detector.

The GC analysis conditions were as follows: the inlet temperature was set at 280° C., while the detector temperature was maintained at 320° ° C. The oven temperature was initially held at 80° C. for 1 minute, then ramped up to 280° C. at a rate of 20° C. min-1, and subsequently held at 300° C. for 10 minutes. The carrier gas used was N2, flowing at a rate of 40.0 mL per minute. The hydrogen flow rate was 45 mL min-1, the air flow rate was 300 mL min-1, and the tail blow flow rate was 25 mL min-1. A 1 μL injection was made using CH2C12 as the solvent, with o-xylene serving as the internal standard.

The present invention employs low-cost methyl glycolate, derived from the semi-hydrogenation of dimethyl oxalate, as a starting material for glycolide synthesis. This approach significantly slashes the glycolide production costs, in contrast to the traditional two-stage method of oligomerization-depolymerization using glycolic acid as the raw material.

One the one hand, the esterification product resulting from the transesterification of high-boiling alcohol with methyl glycolate in this invention exhibits a significantly lower molecular weight (Mw<800 g/mol) when compared to the molecular weight of glycolic acid oligomers typically employed in depolymerization (usually within the range of 4,000 to 30,000 g/mol) for glycolide production. This lower molecular weight equates to reduced viscosity within the reaction system, effectively mitigating issues related to coking during glycolide production. On the other hand, high-purity glycolide is selectively distillated out of system while high-boiling alcohol is simultaneously recovered and recycled, leveraging the differences in boiling points, which is conducive to cost-effective large-scale glycolide production.

### Embodiment 1

The method for synthesizing glycolide in this example comprises two sequential steps:

Step (i): Initially, 16.30 grams of n-docosanol were placed in a 100 mL flask and melted at 60° C. Subsequently, 4.50 grams of methyl glycolate, along with 0.018 grams of stannous octoate, were added into the flask. The mixture in the flask was stirred under atmospheric pressure, and the temperature was gradually raised to 140° C. to facilitate the evaporation of methanol. The reaction was terminated until no methanol was observed to evaporate. The resulting light yellow liquid in the flask was identified as docosyl glycolate.

Step (ii): The esterification product obtained from step (i) was heated to 225° C., and the system's pressure was reduced to 0.5 kPa through evacuation. Subsequently, the colorless distillate was collected and condensed into white crystals upon cooling. The reaction was concluded when no more liquid distilled out. The collected distillate, weighing 2.07 grams, was identified as glycolide. The yield of glycolide was 71.40% with a purity of 98.2%, as confirmed by gas chromatography analysis.

FIG. 2 shows the 1H NMR spectrum of the glycolide prepared in this example. Notably, a single peak is observed at 8=4.95 ppm, indicating that all hydrogen atoms within the sample's molecular structure exist in the same chemical environment. This observation aligns with the fact that glycolide solely comprises a single type of methylene hydrogen atom. Consequently, the 1H NMR spectrum exclusively displays the resonance absorption peak of the methylene hydrogen atom.

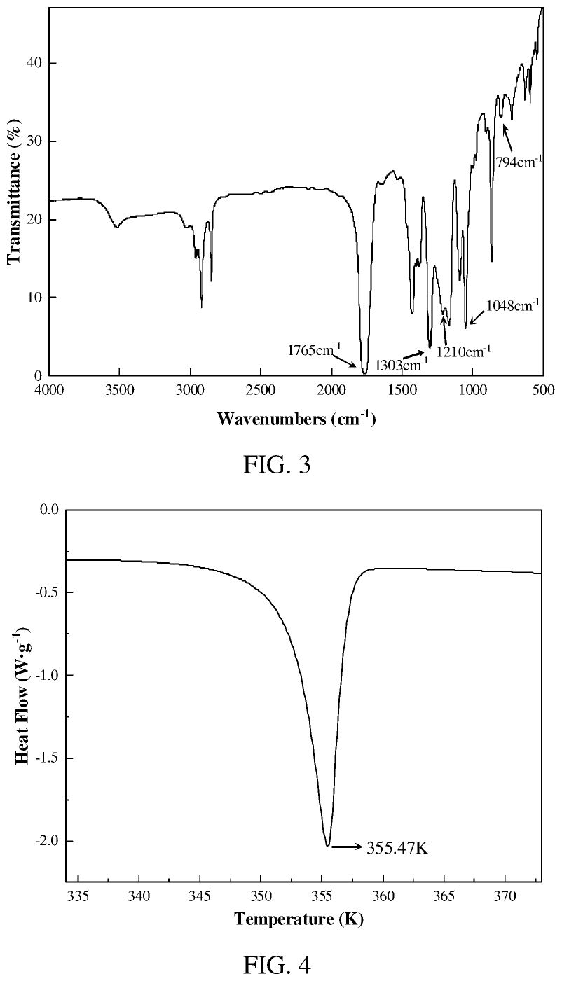

FIG. 3 illustrates the infrared spectrum of the recrystallized glycolide produced in this example, with the horizontal axis representing wave numbers and the vertical axis indicating transmittance. In FIG. 3, the absorption peak of the carbonyl C═O is discerned at 1765 cm⁻¹, while wave numbers 1303 and 1210 cm⁻¹ correspond to the asymmetrical stretching vibration peaks of the ester bond. Additionally, 1048 cm⁻¹ signifies the symmetrical stretching vibration peak of the ester bond, and 794 cm⁻¹ represents the out-of-plane deformation vibration peak of the C—H single bond within the ring structure. The combination of data derived from infrared absorption spectroscopy and the 1H NMR spectrum confirms the product's identity as glycolide.

The melting point of the product was estimated to be 355.47 K according to the DSC profile of the sample obtained in step (ii) (FIG. 4), and the purity of the product (1-X₂) was calculated through Van′t Hoff's rule.

$${T}_{f} = {T}_{0} - \frac{{X}_{2} R {T}_{0}^{2}}{Δ {H}_{f}}$$

wherein, T_{f} is the melting point of the sample to be measured;
T₀ is the melting point of standard glycolide samples;
X₂ is the molar fraction of impurities in the sample to be measured;
ΔH_{f} is the molar enthalpy of melting of the standard sample;
R is the ideal gas constant 8.314 J/mol·K.

Considering the melting point and melting enthalpy (ΔH_{f}) of pure glycolide is reported in literature as 357 K and 13960 J/mol, the measured melting point of glycolide sample in this example (355.47K) allows to calculate the impurity content X₂ according to the above formula as 2.02%. Consequently, the product's purity stands at 97.98%, which is in alignment with the outcome obtained from GC analysis.

### COMPARATIVE EXAMPLE

This comparative example involves the synthesis of glycolide using deploymerization-oligomerization method.

Initially, 35 grams of methyl glycolate were charge into a 250 mL three-neck flask with 0.35 grams of stannous octoate. The mixture was stirred and the temperature was gradually elevated to 160° C. Then nitrogen was used to purge the flask and the temperature was gently raised to 200° ° C. until all methanol had completely evaporated. At this point, a yellow-brown solid had formed on the inner wall of the flask, and stirring was discontinued. The resulting product was identified as glycolic acid oligomer.

The obtained glycolic acid oligomer was then charged into another three-neck flask, and heat it to 240° ° C. while maintaining a vacuum within the reaction system at around 1.5 kPa. A light yellow crystal was distilled out as the crude glycolide, weighing 16.48 g. The purity of crude glycolide was determined as 86.78% through GC analysis.

The obtained crude glycolide was purified by recrystallization using ethyl acetate as solvent. Finally, 12.58 grams of glycolide was obtained with purity of 97.22% and a yield of 55.97% determined by GC analysis.

### Embodiment 2

The method for synthesizing glycolide in this example comprises two sequential steps:

Step (i): Initially, 14.90 grams of n-eicosanoic alcohol were placed in a 100 ml flask and melted at 80° C. Subsequently, 4.50 grams of methyl glycolate, along with 0.018 grams of stannous octoate, were added into the flask. The mixture in the flask was stirred under atmospheric pressure, and the temperature was gradually raised to 140° C. to facilitate the evaporation of methanol. The reaction was terminated when no further methanol was observed to evaporate. The resulting light yellow liquid in the flask was identified as eicosyl glycolate.

Step (ii): The esterification product obtained from step (i) was heated to 210° C., and the system's pressure was lowered to 1.0 kPa through evacuation. Subsequently, the colorless distillate was collected and condensed into white crystals upon cooling. The reaction was concluded when no more liquid was distilled out. The collected distillate, weighing 1.92 grams, was identified as glycolide. The yield of glycolide was 66.20% with a purity of 97.52%, as confirmed by GC analysis.

### Embodiment 3

The method for synthesizing glycolide in this example comprises two sequential steps:

Step (i): Initially, 16.30 grams of n-docosanol were placed in a 100 mL flask and melted at 80° ° C. Subsequently, 4.50 grams of methyl glycolate, along with 0.0675 grams of stannous chloride, were added into the flask. The mixture in the flask was stirred under atmospheric pressure, and the temperature was gradually raised to 160° C. to facilitate the evaporation of methanol. The reaction was terminated when no further methanol was observed to evaporate. The resulting light yellow liquid in the flask was identified as docosyl glycolate.

Step (ii): The esterification product obtained from step (i) was heated to 220° C., and the system's pressure was reduced to 1.5 kPa through evacuation. Subsequently, the colorless distillate was collected and condensed into white crystals upon cooling. The reaction was concluded when no more liquid was distilled out. The collected distillate, weighing 2.04 grams, was identified as glycolide. The yield of glycolide was 70.20% with a purity of 98.34%, as confirmed by GC analysis.

### Embodiment 4

The method for synthesizing glycolide in this example comprises two sequential steps:

Step (i): Initially, 19.10 grams of n-hexcosanol were placed in a 100 mL flask and melted at 80° C. Subsequently, 4.50 grams of methyl glycolate, along with 0.018 grams of zinc oxide, were added into the flask. The mixture in the flask was stirred under atmospheric pressure, and the temperature was gradually raised to 180° C. to facilitate the evaporation of methanol. The reaction was terminated when no further methanol was observed to evaporate. The resulting light yellow liquid in the flask was identified as hexcosyl glycolate.

Step (ii): The esterification product obtained from step (i) was heated to 230° C., and the system's pressure was reduced to 1.0 kPa through evacuation. Subsequently, the colorless distillate was collected and condensed into white crystals upon cooling. The reaction was concluded when no more liquid was distilled out. The collected distillate, weighing 2.05 grams, was identified as glycolide. The yield of glycolide was 70.80% with a purity of 98.24%, as confirmed by GC analysis.

### Embodiment 5

The method for synthesizing glycolide in this example comprises two sequential steps:

Step (i): Initially, 20.50 grams of n-octadecanol alcohol were placed in a 100 mL flask and melted at 80° C. Subsequently, 4.50 grams of methyl glycolate, along with 0.018 grams of antimony trioxide, were added into the flask. The mixture in the flask was stirred under atmospheric pressure, and the temperature was gradually raised to 180° C. to facilitate the evaporation of methanol. The reaction was terminated when no further methanol was observed to evaporate. The resulting light yellow liquid in the flask was identified as octadecyl glycolate.

Step (ii): The esterification product obtained from step (i) was heated to 240° C., and the system's pressure was reduced to 0.5 kPa through evacuation. Subsequently, the colorless distillate was collected and condensed into white crystals upon cooling. The reaction was concluded when no more liquid was distilled out. The collected distillate, weighing 2.06 grams, was identified as glycolide. The yield of glycolide was 71.20% with a purity of 98.02%, as confirmed by GC analysis.

### Embodiment 6

The method for synthesizing glycolide in this example comprises two sequential steps:

Step (i): Initially, 21.90 grams of n-triacontanol were placed in a 100 mL flask and melted at 80° C. Subsequently, 4.50 grams of methyl glycolate, along with 0.018 grams of zinc acetylacetonate, were added into the flask. The mixture in the flask was stirred under atmospheric pressure, and the temperature was gradually raised to 180° C. to facilitate the evaporation of methanol. The reaction was terminated when no further methanol was observed to evaporate. The resulting light yellow liquid in the flask was identified as triacontyl glycolate.

Step (ii): The esterification product obtained from step (i) was heated to 260° C., and the system's pressure was reduced to 1.5 kPa through evacuation. Subsequently, the colorless distillate was collected and condensed into white crystals upon cooling. The reaction was concluded when no more liquid was distilled out. The collected distillate, weighing 2.09 grams, was identified as glycolide. The yield of glycolide was 72.00% with a purity of 97.82%, as confirmed by GC analysis.

### Embodiment 7

The method for synthesizing glycolide in this example involves following steps:

Step (i): Initially, 27.00 grams of n-octadecanol were placed in a 100 mL flask and melted at 60° C. Subsequently, 5.40 grams of methyl glycolate, along with 0.0108 grams of antimony trioxide, were added into the flask. The mixture in the flask was stirred under atmospheric pressure, and the temperature was slowly raised to 100° C. to facilitate the evaporation of methanol. The reaction was terminated when no further methanol was observed to evaporate. The resulting light yellow liquid in the flask was identified as octadecyl glycolate.

Step (ii): The esterification product obtained from step (i) was heated to 200° ° C., and the system's pressure was reduced to 0.5 kPa through evacuation. Subsequently, the colorless distillate was collected and condensed into white crystals upon cooling. The reaction was concluded when no more liquid was distilled out. The collected distillate, weighing 1.13 grams, was identified as glycolide. The yield of glycolide was 32.50% with a purity of 92.36%, as confirmed by GC analysis.

Step (iii): After the distillation residue in the flask from step (ii) had cooled down to 60° C., 5.4 grams of methyl glycolate were recharged into the flask. The mixture was stirred and the temperature was slowly elevated to 100° C. at atmospheric pressure until no more methanol vaporized out, resulting in octadecyl glycolate again. Subsequently, the system's temperature was increased to 200° C., and the pressure was reduced to 0.5 kPa again through evacuation, leading to the collection of glycolide as the distillate.

Step (iii) was repeated four more times, with a total of 27.0 grams of methyl glycolate feed. The cumulative glycolide product after recrystallization amounted to 13.66 grams, achieving an overall yield of 78.48%, while the average glycolide yield for the last four runs of step (iii) was 90.82% with a purity of 98.84% as confirmed by GC analysis.

### Embodiment 8

The method for synthesizing glycolide in this example comprises two sequential steps:

Step (i): Initially, 15.70 grams of 1,20-eicosanediol were placed in a 100 mL flask and melted at 80° C. Subsequently, 18 grams of methyl glycolate, along with 0.072 grams of antimony trioxide, were added into the flask. The mixture in the flask was stirred under atmospheric pressure, and the temperature was gradually raised to 180° C. to facilitate the evaporation of methanol. The reaction was terminated when no further methanol was observed to evaporate. The resulting light yellow liquid in the flask was identified as 1,20-eicosanediyl bis(2-hydroxyacetate).

Step (ii): The esterification product obtained from step (i) was heated to 225° C., and the system's pressure was reduced to 1.0 kPa through evacuation. Subsequently, the colorless distillate was collected and condensed into white crystals upon cooling. The reaction was concluded when no more liquid was distilled out. The collected distillate, weighing 9.05 grams, was identified as glycolide. The yield of glycolide was 78.02% with a purity of 97.67%, as confirmed by GC analysis.

### Embodiment 9

The method for synthesizing glycolide in this example comprises two sequential steps:

Step (i): Initially, 16.50 grams of 3,7,11,15-tetramethyl-1,2,3-hexadecanetriol were placed in a 250 mL flask and melted at 80° C. Subsequently, 67.5 grams of methyl glycolate, along with 0.27 grams of stannous octoate, were added into the flask. The mixture in the flask was stirred under atmospheric pressure, and the temperature was gradually raised to 180ºC to facilitate the evaporation of methanol. The reaction was terminated when no further methanol was observed to evaporate. The resulting light yellow liquid in the flask was identified as 3,7,11,15-tetramethylhexadecane-1,2,3-triyl tris(2-hydroxyacetate).

Step (ii): The esterification product obtained from step (i) was heated to 225° C., and the system's pressure was reduced to 1.5 kPa through evacuation. Subsequently, the colorless distillate was collected and condensed into white crystals upon cooling. The reaction was concluded when no more liquid was distilled out. The collected distillate, weighing 35.67 grams, was identified as glycolide. The yield of glycolide was 82.00% with a purity of 97.35%, as confirmed by GC analysis.

### Embodiment 10

The method for synthesizing glycolide in this example comprises two sequential steps:

Step (i): Initially, 16.30 grams of n-docosanol were placed in a 250 mL flask and melted at 80° C. Subsequently, 90.00 grams of methyl glycolate, along with 0.360 grams of stannous octanoate, were added to the flask. The mixture in the flask was stirred under atmospheric pressure, and the temperature was gradually raised to 180° ° C. to facilitate the evaporation of methanol. The reaction was terminated when no further methanol was observed to evaporate. The resulting light yellow liquid in the flask was identified as docosyl glycolate.

Step (ii): The esterification product obtained from step (i) was heated to 225° C., and the system's pressure was reduced to 1.5 kPa through evacuation. Subsequently, the colorless distillate was collected and condensed into white crystals upon cooling. The reaction was concluded when no more liquid was distilled out. The collected distillate, weighing 54.26 grams, was identified as glycolide. The yield of glycolide was 93.56% with a purity of 98.58%, as confirmed by GC analysis.

### Embodiment 11

The method for synthesizing glycolide in this example comprises two sequential steps:

Step (i): initially, 17.70 grams of docosyl formate were placed in a 100 mL flask and melted at 60° C. Subsequently, 4.50 grams of methyl glycolate, along with 0.0108 grams of stannous chloride, were added into the flask. The mixture in the flask was stirred under atmospheric pressure, and the temperature was gradually raised to 100° C. to facilitate the evaporation of methanol. The reaction was terminated when no further methanol was observed to evaporate. The resulting light yellow liquid in the flask was identified as docosyl glycolate.

Step (ii): The esterification product obtained from step (i) was heated to 200° C., and the system's pressure was reduced to 0.5 kPa through evacuation. Subsequently, the colorless distillate was collected and condensed into white crystals upon cooling. The reaction was concluded when no more liquid was distilled out. The collected distillate, weighing 1.93 grams, was identified as glycolide. The yield of glycolide was 66.20% with a purity of 97.34%, as confirmed by GC analysis.

Step (iii): After the distillation residue in the flask from step (ii) had cooled down to 60° C., another 4.5 grams of methyl glycolate were charged into the flask. The mixture was stirred and the temperature was slowly elevated to 100° C. at atmospheric pressure until no more methanol vaporized out, resulting in docosyl glycolate again. Subsequently, the system's temperature was increased to 200° ° C., and the pressure was reduced to 0.5 kPa again through evacuation, leading to the collection of glycolide as the distillate.

Step (iii) was repeated four more times, with a total feed of 22.5 grams methyl glycolate. The cumulative glycolide product after recrystallization amounted to 12.45 grams, achieving an overall yield of 85.87%, while the average glycolide yield for the last four runs of step (iii) was 90.74% with a purity of 97.94% as confirmed by GC analysis.

As previously stated, the method disclosed in the present invention significantly enhances both the yield and purity of glycolide during its synthesis from methyl glycolate. This enhancement is accomplished by introducing a high-boiling alcohol into the reaction system and conducting the synthesis entirely in a low-viscosity liquid phase, which promotes efficient heat transfer and avoids the issue of coking typically encountered in traditional glycolide synthesis routes. Consequently, this method offers a significant advantage in the large-scale industrial production of glycolide, enabling the continuous production of high-purity glycolide without the need for additional separation and purification steps.

The examples described herein represent the preferred embodiments for implementing the current invention. It should be noted that these embodiments are intended to describe and not to limit the disclosure. For those skilled in the art, a number of improvements and substitutions can be made without departing from the technical principles of the present invention, and these improvements and substitutions should also be regarded as within the scope of protection for this invention.

1. A method for synthesizing glycolide using methyl glycolate as a raw material, comprising two steps as illustrated in the following reaction formulas: 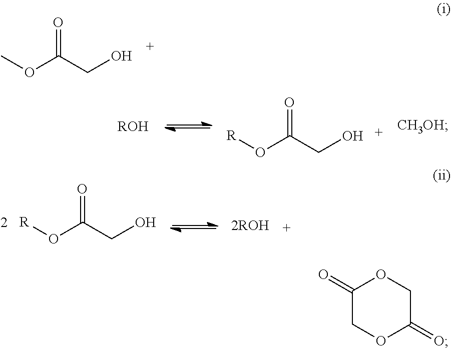
wherein, the ROH represents a straight/branched chain alcohol, acid or ester containing 18-30 carbons and at least one hydroxyl group, and the ROH obtained in step (ii) is recycled.

2. The method of claim 1, wherein the reaction of step (i) is conducted with a catalyst at a temperature ranging from 100 to 180° C.

3. The method of claim 2, wherein the said catalyst is selected from stannous octoate, stannous chloride, zinc oxide, antimony oxide, and zinc acetylacetonate.

4. The method of claim 2, wherein the mass ratio of the catalyst to methyl glycolate is in the range of 0.2 to 1.5 wt %.

5. The method of claim 1, wherein the molar ratio of the hydroxyl group present in ROH to methyl glycolate fed in the reaction of step (i) ranges from 1:0.6 to 1:20.

6. The method of claim 1, wherein the reaction in the said step (ii) is conducted under vacuum distillation, with a pressure ranging from 0.5 to 1.5 kPa and a temperature ranging from 200 to 260° C.

7. The method of claim 1, wherein the boiling point of said ROH is in the range of 330 to 460° C.

8. A method for synthesizing glycolide, wherein methyl glycolate is used as raw material and the glycolide is obtained through the following reaction loop, comprising the recyclable steps of: 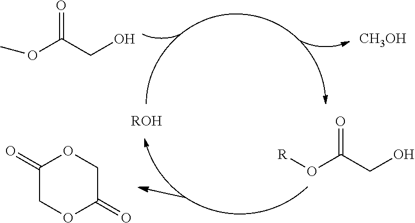
(i) the raw material methyl glycolate undergoing a transesterification reaction with ROH between 100 to 180° C., and obtained methanol distilled out of system, while esterified product retained in the system;
(ii) the esterification product of step (i) heated up to 200 to 260° C. at a pressure of 0.5 kPa to 1.5 kPa to obtain the glycolide and ROH, wherein the glycolide is distilled out of the system, while ROH is retained in the system for recycling;
wherein, the ROH represents a straight/branched chain alcohol, acid or ester containing 18-30 carbons and at least one hydroxyl group, with a boiling point between 330 and 460° C.

9. The method of claim 8, wherein the reaction in step (i) is conducted with a catalyst selected from stannous octoate, stannous chloride, zinc oxide, antimony oxide, and zinc acetylacetonate.

10. The method of claim 8, wherein the mass ratio of the catalyst to methyl glycolate is in the range of 0.2 to 1.5 wt %.

11. The method of claim 8, wherein the molar ratio of the hydroxyl group contained in ROH to methyl glycolate fed in the reaction of step (i) ranges from 1:0.6 to 1:20.
